(19) United States
(12) Patent Application Publication
(10) Pub. No.: US 2010/0168579 A1
(43) Pub. Date: Jul. 1, 2010

(54) METHODS AND APPARATUS FOR HIGH SPEED IMAGE ACQUISITION RATES IN 3D MEDICAL IMAGING

(75) Inventor: Dave Prater, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/445,799

(22) PCT Filed: Oct. 18, 2007
(86) PCT No.: PCT/IB07/54253

§ 371 Date: Apr. 16, 2009

Related U.S. Application Data
(60) Provisional application No. 60/862,316, filed on Oct. 20, 2006.

(51) Int. Cl. A61B 8/14

(52) U.S. Cl. 600/447

(57) ABSTRACT

Methods and apparatus for high speed image acquisition rates in medical imaging are provided. An embodiment includes scanning an R-theta sector slice, wherein R denotes radius of the sector and theta denotes angle of the sector; varying a value of each of phi and theta, such that phi denotes an elevation location of the R-theta slice, and theta is a function of phi; and, iterating the scanning, acquiring by this method at least one image at an improved acquisition rate compared to that of scanning methods using a constant value of theta.

The technical field of the application is methods and apparatus for high speed image acquisition rates for 3D medical imaging.

Ultrasound is a noninvasive, easily portable and relatively inexpensive tool for medical imaging and diagnosis. Ultrasound waves emitted by a transducer are reflected at tissue boundaries and recorded by a probe, allowing a user to detect boundaries of anatomical structures with different sonic impedance. Bonneau et al. (2003), Adaptive Volume Construction from Ultrasound Images of a Human Heart, Joint EUROGRAPHICS-IEEE TCVG Symposium on Visualization, herein incorporated by reference. The cost, noninvasiveness and portability of ultrasound have led to the wide scale adoption of ultrasound imaging of a variety of organs. In particular, ultrasound is often used to detect motion, for example, the movement of a fetus or an organ such as the heart, to detect potential pathologies. Such imaging of the heart, referred to as echocardiography, poses particular challenges because diagnosis of some heart problems, such as mitral valve prolapse, involves analyzing movements of the heart that are subtle and can be detected only if the imaging device can acquire images rapidly.

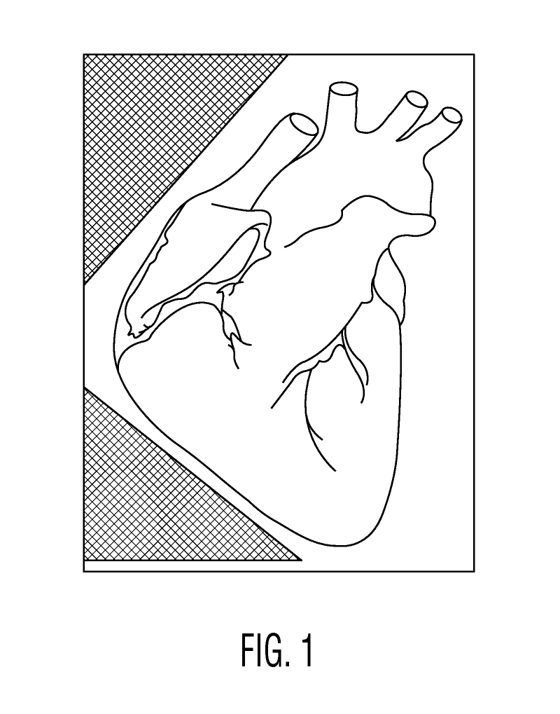

In conventional 3D volume scanning, the typical pattern is to scan a series of R-theta sector slices, with R denoting the radius of the sector and theta denoting the angle of the sector. Each sector slice is located at a different elevation position. This elevation position will be referred to as the “phi” location. In this conventional 3D volume, the theta value of each slice is constant. The pattern that results when the scan is viewed in the phi-theta plane is a rectangle. (This pattern will be referred to as a “rectangular scanning pattern.”) For cardiac scanning of the left ventricle of the heart from the apical position, use of a rectangular scanning pattern is inefficient as the left ventricle is elliptical (in many cases approximately circular) in the phi-theta plane. This inefficient aspect of conventional 3D volume scanning patterns in echocardiography is illustrated in FIG. 1. Inefficient image acquisition causes lower than desired image acquisition rates.

Accordingly, an embodiment of the invention provided herein is a method of scanning of a medical volume of interest in a subject, including scanning an R-theta sector slice. R denotes radius of the sector and theta denotes angle of the sector. Theta is a function of phi, and phi denotes an elevation location of the R-theta slice. The scanning is performed at first values of phi and theta. The method also includes varying phi and theta to at least second values of phi and theta. The method also includes iterating the scanning at the varied values acquires at least one image at an improved acquisition rate compared to that of scanning methods using a constant value of theta.

In a further embodiment, the function is a piecewise, stepwise or “case by case” function. In another embodiment, the function is a discontinuous function.

In another embodiment, the function results in values of theta such that a scanning pattern, when viewed from a phi-theta plane, is a conic section, such as an ellipse. In another embodiment, the function results in values of theta such that a scanning pattern, when viewed from the phi-theta plane, is a polygon.

Another embodiment of the invention provides a medical imaging device for scanning a volume of interest in a subject. The device is designed to scan an R-theta sector slice, wherein R denotes radius of the sector, theta is a function of phi and denotes angle of the sector, and phi denotes an elevation location of the R-theta slice. The device is further designed to vary phi and theta over a plurality of values of phi and theta and iterate the scanning at the plurality of resulting varied values of phi and theta, wherein the device acquires at least one image at an improved acquisition rate compared to that of scanning methods using a constant value of theta.

In a further embodiment, at least one from the plurality of functions is selected to generate a desired pattern for scanning the volume of interest. In another embodiment, a volume of interest to be scanned is selected by a user. In a further embodiment, the user selects the function that is appropriate for the desired volume of interest. In another embodiment, the device detects a volume of interest that is being scanned and selects the function that yields a high image acquisition rate for the desired volume of interest. In another embodiment, the device detects the boundaries of a scanned volume and produces a scanning pattern comprising the boundaries of the volume.

In another embodiment, the medical imaging device is an ultrasound imaging device. In a further embodiment, the medical imaging device is a three-dimensional ultrasound scanner. In another embodiment, the medical imaging device is a three-dimensional cardiac ultrasound scanner.

FIG. 1 is a drawing showing scanning of a heart using a conventional rectangular scanning pattern.

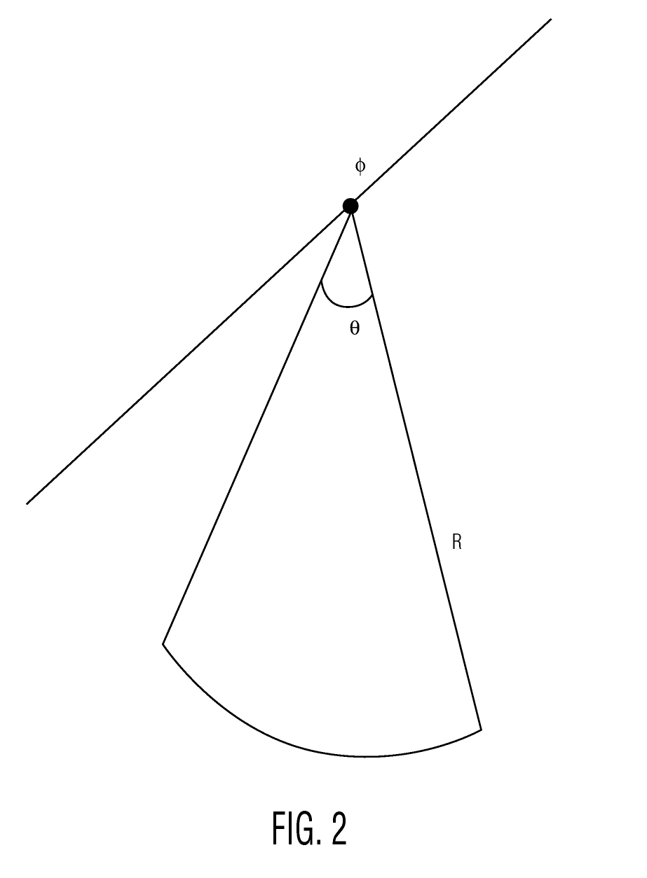

FIG. 2 is a representation of three-dimensional scanning parameters.

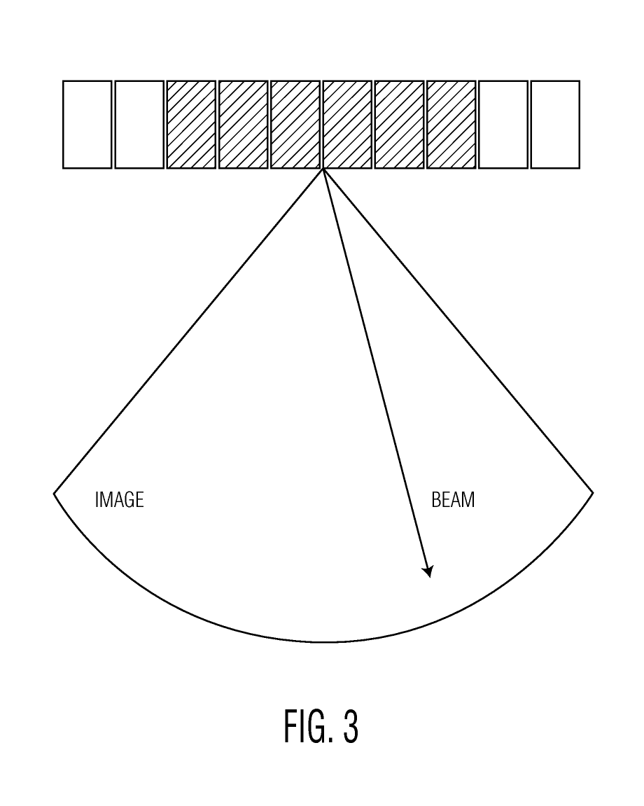

FIG. 3 shows an implementation of sector scanning using an array of ultrasound elements.

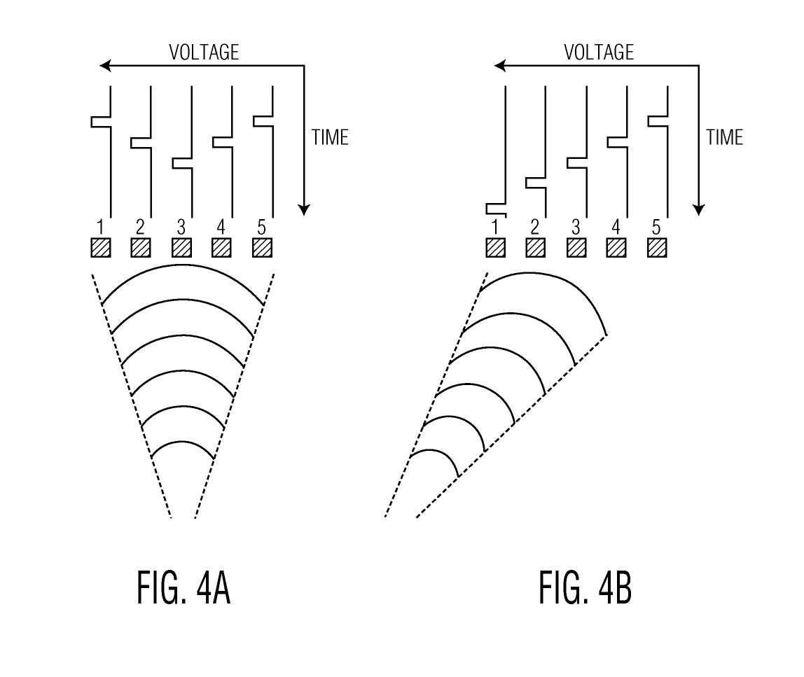

FIG. 4 is a set of drawings that show the formation and steering of beams through use of an array of elements.

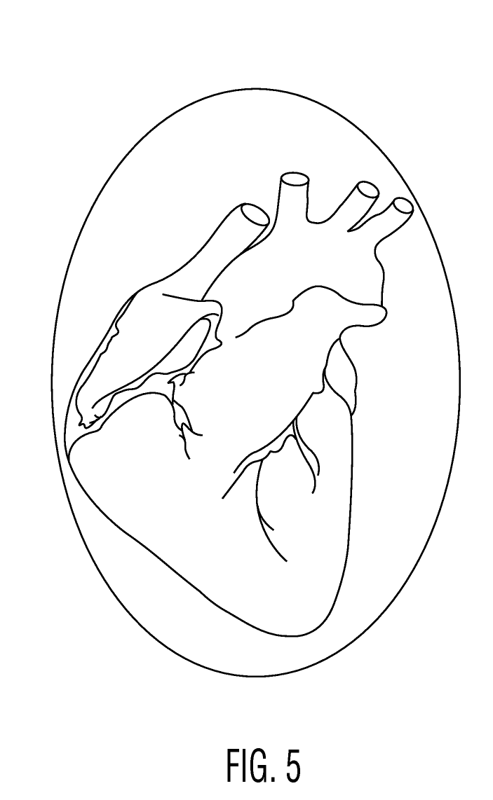

FIG. 5 is a drawing showing an elliptical scanning pattern.

The methods and apparatus herein are applied to ultrasound scanning applications, such as cardiac scanning, however, other medical imaging devices and other target organs are within the scope of the invention.

Ultrasound imaging systems include a transducer that acts as both a transmitter and a receiver. The transducer consists of either a single element or an array of multiple elements. Ultrasound elements may be made of several materials including, but not limited to: piezocrystals, lead zirconate titanate (PZT), piezo-electric material, and piezo-composite material. In transmission mode, the transducer converts electrical signals into mechanical vibrations, which are transmitted into the body as ultrasound waves. In reception mode, the echoes (backscatter) of the ultrasound waves are converted into electrical signals, then processed.

Conventional ultrasound scanning methods have used rectangular scanning patterns. As shown in FIG. 1, conventional rectangular scanning patterns are inefficient for echocardiography because the heart is not rectangular, and unwanted regions of the body are included when a desired organ is imaged. Examples of these unwanted regions are represented schematically in FIG. 1 by crosshatched triangles.

FIG. 2 is a representation of three-dimensional scanning parameters. The process of 3D volume scanning involves acquiring and assembling multiple sector scans, taken at different phi (indicated in the Figure by the Greek symbol, Φ) values. The parameters phi (Φ), theta (θ) and R are modified to adjust the size, shape, position and orientation of the 3D image. In conventional rectangular scanning patterns, theta is constant as a function of phi. In embodiments of the invention provided herein, the value of theta during scanning is changed as a function of phi to produce patterns of various shapes such as an ellipse, and of various sizes as viewed in the phi-theta plane. Frame tables can be modified in order to account for the theta extent of each slice being dependent on the phi location.

An implementation of sector scanning with an array of ultrasound elements is shown in FIG. 3. The array of elements is represented by a row of rectangles, and excited elements are represented by shaded rectangles. One or more elements are excited to form a beam. Sector scanning is also possible by mechanically steering a single element so that the focus of the element is in the desired direction. This beam (e.g. an ultrasound beam) travels through the body in the specified direction and the echoes are received by the transducer. This process is repeated to create an image of a volume of interest.

Beams are formed and steered through constructive interference and the controlling of the phasing, i.e. the relative phase shift in superimposing waves, of various elements. Constructive interference occurs when the summation or combination of two waves results in a wave with an amplitude greater than that of the individual waves. FIG. 4, panel A shows the focusing of a beam by controlling of the timing of the exciting of elements as is well known in the practice of phased ultrasound array. Elements 1 and 5 are excited first, followed by elements 2 and 4, then element 3. FIG. 4, panel B shows the steering of a beam to one side, e.g. the left, by exciting element 5, then element 4, then element 3, then element 2, and finally element 1. Webb, Introduction to Biomedical Imaging 126-28 (2003).

In 3D volume scanning, a typical pattern is to scan a series of R-theta sector slices with each slice located at a different phi location. In conventional scanning as performed heretofore, the theta extent of each slice is constant. The pattern that results from scanning with a constant value of theta is a rectangle when viewed in the phi-theta plane. For cardiac scanning of the left ventricle from the apical position, rectangular scanning is inefficient as the left ventricle is elliptical (in many cases approximately circular) in the phi-theta plane. This inefficient aspect of conventional 3D volume scanning patterns in echocardiography is illustrated in FIG. 1. The cross-hatched triangles in FIG. 1 represent volumes outside of the volume of interest (i.e. the heart) that were unnecessarily scanned. A result of scanning outside of the volume of interest is increases in the time required for a scanning and decreases in the efficiency of the scan. Thus, inefficient image acquisition causes lower than desired image acquisition rates.

To improve efficiency, various embodiments of the invention provided herein employ elliptical 3D scanning Thus the volume rate in 3D scanning is increased with little or no loss in diagnostic utility, for example, when scanning the left ventricle in the apical view.

Various embodiments of the invention provided herein improve image acquisition rates by modifying a scanning pattern such that theta is made a function of phi. Accordingly, elliptical scanning patterns are facilitated which, as shown in FIG. 5, image the heart more efficiently because the boundaries of the imaging pattern approximate the shape of the heart more closely than a conventional rectangular scanning pattern. In addition to elliptical scanning patterns, other geometric functions may be implemented in order to image organs of varying shapes, for example, functions that produce patterns including but not limited to conic sections and polygons.

For applications in which there is a need to maintain a uniformity of scanning time for each of a plurality of sub-volumes within the volume of interest, the same number of scan lines for each sub-volume may be useful. As the sub-volumes are no longer rectangular in the theta-phi plane, hence a more complex shape for each sub-volume is needed. The more complex shapes allow for a pattern at the sub-volume interface line that has the effect that it lessens a temporary and unwanted artifact sometimes observed along the interface line.

Additional embodiments of the invention provided herein include medical imaging devices to implement the above methods in a three-dimensional ultrasound scanner for cardiac or other organs. Some embodiments of such a device allow the user to select a function from a plurality of functions to generate a desired pattern for scanning the volume of interest. Related embodiments of these devices select an appropriate function for efficient scanning of the desired volume of interest and/or detect the boundaries of the volume of interest and design a function that produces an efficient scanning function for the volume of interest.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting.

What is claimed is:

1. A method of scanning at an improved acquisition rate a volume of interest in a subject, the method comprising:
scanning an R-theta sector slice, wherein R denotes radius of the sector, theta denotes angle of the sector and is a function of phi, and phi denotes an elevation location of the R-theta slice, wherein the scanning is performed at first values of phi and theta;
varying phi and theta to at least second values of phi and theta, wherein iterating the scanning at the varied values acquires at least one image at an improved acquisition rate compared to that of scanning methods using a constant value of theta.

2. The method of claim 1, wherein the function is a piecewise function.

3. The method of claim 1, wherein the function is a discontinuous function.

4. The method of claim 1, wherein the function results in values of theta such that a scanning pattern, when viewed from a phi-theta plane, is a conic section.

5. The method of claim 4, wherein the conic section is an ellipse.

6. The method of claim 1, wherein the function results in values of theta such that a scanning pattern, when viewed from the phi-theta plane, is a polygon.

7. A medical imaging device for scanning a volume of interest in a subject, the device designed to scan an R-theta sector slice, wherein R denotes radius of the sector, theta is a function of phi and denotes angle of the sector, and phi denotes an elevation location of the R-theta slice; and further designed to vary phi and theta over a plurality of values of phi and theta and iterate the scanning at the plurality of resulting varied values of phi and theta, wherein the device acquires at least one image at an improved acquisition rate compared to that of scanning methods using a constant value of theta.

8. The device according to claim 7, wherein at least one from the plurality of functions is selected to generate a desired pattern for scanning the volume of interest.

9. The device according to claim 7, wherein the volume of interest to be scanned is selected by a user.

10. The device according to claim 9, wherein the user selects a function that yields the highest image acquisition rate for the desired volume of interest.

11. The device according to claim 7, wherein the device detects a volume of interest that is being scanned and selects a function that yields a high image acquisition rate for the desired volume of interest.

12. The device according to claim 7, wherein the device detects the boundaries of a scanned volume and produces a scanning pattern comprising the boundaries of the volume.

13. The medical imaging device according to claim 7, wherein the device is an ultrasound imaging device.

14. The medical imaging device according to claim 13, wherein the device is a three-dimensional ultrasound scanner.

15. The medical imaging device according to claim 13, wherein the device is a three-dimensional cardiac ultrasound scanner.
